(19) United States
(12) Patent Application Publication
(10) Pub. No.: US 2013/0078681 A1
(43) Pub. Date: Mar. 28, 2013

(54) METHOD FOR PRODUCING AN L-AMINO ACID USING A BACTERIUM OF THE ENTEROBACTERIACEAE FAMILY HAVING ATTENUATED EXPRESSION OF GENES ENCODING A LYSINE/ARGININE/ ORNITHINE TRANSPORTER

(71) Applicant: AJINOMOTO CO., INC., Tokyo (JP)

(72) Inventors: Yulia Georgievna Rostova, Moscow (RU); Elvira Borisovna Voroshilova, Moscow (RU); Mikhail Markovich Gusyatiner, Moscow (RU)

(73) Assignee: AJINOMOTO CO., INC., Tokyo (JP)

(21) Appl. No.: 13/687,353

(22) Filed: Nov. 28, 2012

Related U.S. Application Data
(63) Continuation of application No. PCT/JP2011/063304, filed on Jun. 3, 2011.

(30) Foreign Application Priority Data
Jun. 3, 2010 (RU) 2010122646

(51) Int. Cl. C12P 13/10; C12N 15/70; C12P 13/08

(52) U.S. Cl. CPC C12P 13/10; C12P 13/08; C12N 15/70
USPC 435/114; 435/252.3; 435/115; 435/252.33

(57) ABSTRACT

The present invention provides a method for producing an L-amino acid using a bacterium of the Enterobacteriaceae family, particularly a bacterium belonging to genus Escherichia or Pantoea, in which expression of a gene encoding a lysine/arginine/ornithine transporter has been attenuated.

This application is a Continuation of, and claims priority under 35 U.S.C. §120 to, International Application No. PCT/JP2011/063304, filed Jun. 3, 2011, and claims priority therethrough under 35 U.S.C. §119 to Russian Patent Application No. 2010122646, filed Jun. 3, 2010, the entireties of which are incorporated by reference herein. Also, the Sequence Listing filed electronically herewith is hereby incorporated by reference (File name: 2012-11-28T_US-430_Seq_List; File size: 31 KB; Date recorded: Nov. 28, 2012).

### BACKGROUND OF THE INVENTION

1. Field of the Invention

The present invention relates to the microbiological industry, and specifically to a method for producing an L-amino acid using a bacterium of the Enterobacteriaceae family in which expression of one or more genes encoding a lysine/arginine/ornithine transporter are attenuated.

2. Brief Description of the Related Art

Conventionally, L-amino acids are industrially produced by fermentation methods utilizing strains of microorganisms obtained from natural sources, or mutants thereof. Typically, the microorganisms are modified to enhance production yields of L-amino acids.

Many techniques to enhance L-amino acid production yields have been reported, including by transforming microorganisms with recombinant DNA (see, for example, U.S. Pat. No. 4,278,765). Other techniques for enhancing production yields include increasing the activities of enzymes involved in amino acid biosynthesis and/or desensitizing the target enzymes of the feedback inhibition caused by the resulting L-amino acid (see, for example, WO 95/16042 or U.S. Pat. Nos. 4,346,170; 5,661,012 and 6,040,160).

Another way to enhance L-amino acid production yields is to attenuate expression of a gene or several genes involved in the degradation of the target L-amino acid, genes diverting the precursors of the target L-amino acid from the L-amino acid biosynthetic pathway, genes involved in the redistribution of carbon, nitrogen, and phosphate fluxes, and genes coding for toxins etc.

The hisJ and argT genes of Salmonella typhimurium encode two periplasmic binding proteins, J and LAO, which are involved in histidine and arginine transport, respectively, and which interact with a common membrane-bound component, the P protein. The complete nucleotide sequences of these two genes have been determined. The two genes show extensive homology (70%) and presumably arose by tandem duplication of a single ancestral gene. The two encoded proteins now perform distinct functions but still retain sufficient homology to permit interaction with the same site on the membrane-bound P protein (Higgins C. F., Ames G. F., Proc Natl Acad Sci USA.; 78(10):6038-42(1981)).

The superfamily of traffic ATPases (ABC transporters) includes bacterial periplasmic transport systems (permeases) and various eukaryotic transporters. The histidine permease of S. typhimurium and Escherichia coli is composed of a soluble receptor including a membrane-bound complex containing four subunits, the substrate-binding protein (HisJ), and is energized by ATP. It was shown that histidine transport depends entirely on both ATP and the ligand HisJ, and is affected by pH, temperature, and salt concentration. Transport is irreversible and accumulation reaches a plateau at which point transport ceases. The permease is inhibited by ADP and by high concentrations of internal histidine. The inhibition by histidine likely indicates that the membrane-bound complex HisQ/M/P includes a substrate-binding site. The reconstituted permease activity corresponds to an about 40-70% turnover rate relative to the in vivo rate of transport (Liu C. E., Ames G. F., J Biol Chem.; 272(2):859-66(1997)).

The membrane-bound complex of the Salmonella typhimurium periplasmic histidine permease is composed of two integral membrane proteins, HisQ and HisM, and two copies of an ATP-binding subunit, HisP. The complex hydrolyzes ATP upon induction of the activity by the soluble receptor and the periplasmic histidine-binding protein, HisJ. It was shown that the nucleotide-binding component can be stripped off of the integral membrane components, HisQ and HisM. The complex can be reconstituted by using the HisP-depleted membranes containing HisQ and HisM and pure soluble HisP. HisP was shown to have high affinity for the HisP-depleted complex, HisQM, and two HisP molecules are recruited independently of each other for each HisQM unit. The in vitro reassembled complex has entirely normal properties, and responds to the HisJ and ATPase inhibitors with the same characteristics as the original complex and in contrast to those of soluble HisP. These results show that HisP is absolutely required for ATP hydrolysis, HisQM cannot hydrolyze ATP, HisP depends on HisQM to relay the inducing signal from the soluble receptor HisJ, and HisQM regulates the ATPase activity of HisP. It was also shown that HisP changes conformation upon exposure to phospholipids (Liu P. Q., Ames G. F., Proc Natl Acad Sci USA.; 95(7):3495-500(1998)).

The ArgT protein of E. coli can exist as a cytoplasmic precursor protein of 28 kDa and/or a mature periplasmic protein of 25.8 kDa. To elucidate the involvement of proteolysis in the regulation of stationary-phase adaptation, the clpA, clpX, and clpP protease mutants of Escherichia coli were subjected to proteome analysis during growth and carbon starvation. The periplasmic lysine-arginine-ornithine binding protein ArgT in the clpA, clpX, or clpP mutant did not display induction typical of late-stationary-phase wild-type cells (Weichart D. et al., Journal of Bacteriology, Vol. 185, No. 1, p. 115-125(2003)).

But currently, there have been no reports of attenuating expression of genes encoding a lysine/arginine/ornithine transporter for the purpose of producing L-amino acids.

### SUMMARY OF THE INVENTION

Aspects of the present invention include enhancing the productivity of L-amino acid-producing strains and providing a method for producing an L-amino acid using these strains.

It has been found that attenuating expression of the argT-hisJQMP cluster can enhance production of amino acids, for example, diaminomonocarboxylic acids, such as L-lysine, L-arginine, ornithine, and citrulline.

The present invention provides a bacterium of the Enterobacteriaceae family having an increased ability to produce amino acids, for example, diaminomonocarboxylic acids, such as L-lysine, L-arginine, ornithine, and citrulline.

It is an aspect of the present invention to provide an L-amino acid-producing bacterium of the Enterobacteriaceae family, wherein said bacterium has been modified to attenuate expression of one or more genes encoding a lysine/arginine/ornithine transporter.

It is a further aspect of the present invention to provide the bacterium as described above, wherein said one or more genes encoding a lysine/arginine/ornithine transporter is/are one or more genes of the argT-hisJQMP cluster.

It is a further aspect of the present invention to provide the bacterium as described above, wherein expression of said one or more genes encoding a lysine/arginine/ornithine transporter is/are attenuated by inactivation of said one or more genes.

It is a further aspect of the present invention to provide the bacterium as described above, wherein said one or more genes of the argT-hisJQMP cluster is/are inactivated.

It is a further aspect of the present invention to provide the bacterium as described above, wherein said bacterium belongs to genus Escherichia.

It is a further aspect of the present invention to provide the bacterium as described above, wherein said bacterium is Escherichia coli.

It is a further aspect of the present invention to provide the bacterium as described above, wherein said bacterium belongs to genus Pantoea.

It is a further aspect of the present invention to provide the bacterium as described above, wherein said L-amino acid is a diaminomonocarboxylic acid.

It is a further aspect of the present invention to provide the bacterium as described above, wherein said diaminomonocarboxylic acid is selected from the group consisting of L-lysine, L-arginine, ornithine, and citrulline.

It is a further aspect of the present invention to provide a method for producing an L-amino acid comprising:
cultivating the bacterium as described above in a medium, and
collecting said L-amino acid from the medium.

It is a further aspect of the present invention to provide the bacterium as described above, wherein said L-amino acid is a diaminomonocarboxylic acid.

It is a further aspect of the present invention to provide the bacterium as described above, wherein said diaminomonocarboxylic acid is selected from the group consisting of L-lysine, L-arginine, ornithine, and citrulline.

### DESCRIPTION OF THE PREFERRED EMBODIMENTS

The present invention is described in detail below.

1. Bacterium

The bacterium in accordance with the presently disclosed subject matter is a bacterium of the Enterobacteriaceae family which is able to produce an L-amino acid such as a diaminomonocarboxylic acid, wherein the baccterium has been modified to attenuate expression of one or more genes encoding a lysine/arginine/ornithine transporter.

The phrase “bacterium producing an L-amino acid” can mean a bacterium which has an ability to produce and excrete an L-amino acid into a medium, when the bacterium is cultured in the medium.

The term “bacterium producing an L-amino acid” can also mean a bacterium which is able to produce and cause accumulation of an L-amino acid such as diaminomonocarboxylic acid in a culture medium in an amount larger than a wild-type or parental strain of E. coli, such as E. coli K-12, and can also mean that the bacterium is able to cause accumulation in a medium of an amount not less than 0.5 g/L, or not less than 1.0 g/L in another example, of the target L-amino acid.

The term “diaminomonocarboxylic acid” can include L-lysine, L-arginine, ornithine, and citrulline. These amino acids have a positive charge due to the presence of an amino group.

The Enterobacteriaceae family includes bacteria belonging to the genera Escherichia, Enterobacter, Erwinia, Klebsiella, Pantoea, Photorhabdus, Providencia, Salmonella, Serratia, Shigella, Morganella, Yersinia, etc. Specifically, those classified into the Enterobacteriaceae according to the taxonomy used by the NCBI (National Center for Biotechnology Information) database (www.ncbi.nlm.nih.gov/Taxonomy/Browser/wwwtax.cgi?id=543) can be used. A bacterium belonging to the genus Escherichia or Pantoea is preferred.

The phrase “a bacterium belonging to the genus Escherichia” can mean that the bacterium is classified into the genus Escherichia according to the classification known to a person skilled in the art of microbiology. Examples of a bacterium belonging to the genus Escherichia include, but are not limited to, Escherichia coli (E. coli).

The bacterium belonging to the genus Escherichia is not particularly limited; however, e.g., bacteria described by Neidhardt, F. C. et al. (Escherichia coli and Salmonella typhimurium, American Society for Microbiology, Washington D.C., 1208, Table 1) can be used.

The phrase “a bacterium belonging to the genus Pantoea” can mean that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii or the like, based on the nucleotide sequence analysis of 16S rRNA, etc. (Int. J. Syst. Bacteriol., 43, 162-173 (1993)).

The phrase “expression of one or more genes encoding a lysine/arginine/ornithine transporter is attenuated” means that the bacterium has been modified in such a way that the modified bacterium contains reduced amounts, or even none, of the lysine/arginine/ornithine transporter as compared with an unmodified bacterium, or the modified bacterium is unable to synthesize a lysine/arginine/ornithine transporter.

The phrase “inactivation of a gene” can mean that the modified gene encodes a completely inactive protein. It is also possible that the modified DNA region is unable to naturally express the gene due to the deletion of the gene, the shifting of the reading frame of the gene, the introduction of missense/nonsense mutation(s), or the modification of an adjacent region of the gene, including sequences controlling gene expression, such as promoter(s), enhancer(s), attenuator(s), ribosome-binding site(s), etc.

The presence or absence of a target gene on the chromosome of a bacterium can be detected by well-known methods, including PCR, Southern blotting, and the like. In addition, the level of expression of the gene can be estimated by measuring the amount of mRNA transcribed from the gene using various well-known methods, including Northern blotting, quantitative RT-PCR, and the like. The amount or molecular weight of the protein encoded by the target gene can be measured by well-known methods, including SDS-PAGE followed by immunoblotting assay (Western blotting analysis), and the like.

Examles of one or more genes encoding a lysine/arginine/ornithine transporter include the genes which make up the argT-hisJQMP cluster.

The argT gene (synonyms—ECK2304, b2310) encodes ArgT, which is a subunit of a lysine/arginine/ornithine ABC transporter (synonym—B2310). The argT gene of E. coli (nucleotides complementary to nucleotides 2,425,031 to 2,425,813 in the GenBank accession number NC_{—}000913.2; gi:16130244) is located between the gene hisJ and the gene ubiX, both oriented in the same direction as argT, on the chromosome of E. coli strain K-12. The nucleotide sequence of the argT gene and the amino acid sequence of ArgT encoded by the argT gene are shown in SEQ ID NO: 1 and SEQ ID NO: 2, respectively.

The hisJ gene (synonyms—ECK2303, b2309) encodes HisJ, which is a subunit of the histidine ABC transporter (synonym—B2309). The hist gene of E. coli (nucleotides complementary to nucleotides 2,424,028 to 2,424,810 in the GenBank accession number NC_{—}000913.2; gi: 16130244) is located between the gene argT and the gene hisQ, both oriented in the same direction as hisJ, on the chromosome of E. coli strain K-12. The nucleotide sequence of the hisJ gene and the amino acid sequence of HisJ encoded by the hisJ gene are shown in SEQ ID NO: 3 and SEQ ID NO: 4, respectively.

Attenuation of expression of the hisJ gene is not essential for the present invention, but expression the hisJ gene can be attenuated.

The hisQ gene (synonyms—ECK2302, b2308) encodes HisQ, which is a subunit of the lysine/arginine/ornithine ABC transporter (synonym—B2308). The hisQ gene of E. coli (nucleotides complementary to nucleotides 2,423,252 to 2,423,938 in the GenBank accession number NC_{—}000913.2; gi: 16130243) is located between the gene hisJ and the gene hisM, both oriented in the same direction as hisQ, on the chromosome of E. coli strain K-12. The nucleotide sequence of the hisQ gene and the amino acid sequence of HisQ encoded by the hisQ gene are shown in SEQ ID NO: 5 and SEQ ID NO: 6, respectively.

The hisM gene (synonyms—ECK2301, b2307) encodes HisM, which is a subunit of the lysine/arginine/ornithine ABC transporter (synonym—B2307). The hisM gene of E. coli (nucleotides complementary to nucleotides 2,422,539 to 2,423,255 in the GenBank accession number NC_{—}000913.2; gi: 16130242) is located between the gene hisQ and the gene hisP, both oriented in the same direction as hisM, on the chromosome of E. coli strain K-12. The nucleotide sequence of the hisM gene and the amino acid sequence of HisM encoded by the hisM gene are shown in SEQ ID NO: 7 and SEQ ID NO: 8, respectively.

The hisP gene (synonyms—ECK2300, b2306) encodes HisP, which is a subunit of the lysine/arginine/ornithine ABC transporter (synonym—B2306). The hisP gene of E. coli (nucleotides complemented to nucleotides 2,421,758 to 2,422,531 in the GenBank accession number NC_{—}000913.2; gi: 16130241) is located between the gene hisM and the ORF yfcI, both oriented in the same directions as hisP, on the chromosome of E. coli strain K-12. The nucleotide sequence of the hisP gene and the amino acid sequence of HisP encoded by the hisP gene are shown in SEQ ID NO: 9 and SEQ ID NO: 10, respectively.

Since there may be some differences in DNA sequences between the genera or strains of the Enterobacteriaceae family, the argT-hisJQMP cluster is not limited to the genes shown in SEQ ID NO: 1, SEQ ID NO: 3, SEQ ID NO: 5, SEQ ID NO: 7 and SEQ ID NO: 9, but may include genes homologous to SEQ ID NO: 1, SEQ ID NO: 3, SEQ ID NO: 5, SEQ ID NO: 7 and SEQ ID NO: 9 which encode variant proteins of the ArgT, HisJ, HisQ, HisM and HisP. The phrase “variant protein” can mean a protein which has changes in the sequence, whether they are deletions, insertions, additions, or substitutions of amino acids, but the function of the protein is maintained. The number of changes in the variant protein depends on the position in the three dimensional structure of the protein or the type of amino acid residues. It may be 1 to 30, or in another example 1 to 15, and or in another example 1 to 5 in SEQ ID NO: 2, SEQ ID NO: 4, SEQ ID NO: 6, SEQ ID NO: 8 and SEQ ID NO: 10. These changes can occur in regions of the protein which are not critical for the function of the protein. This is because some amino acids have high homology to one another so the three dimensional structure or activity is not affected by such a change. Therefore, the protein variant encoded by the gene of argT-hisJQMP cluster may be one which has a homology of not less than 80%, or in another example not less than 90%, not less than 95%, in another example not less than 98%, and in another example not less than 99%, with respect to the entire amino acid sequence shown in SEQ ID NO: 2, SEQ ID NO: 4, SEQ ID NO: 6, SEQ ID NO: 8 or SEQ ID NO: 10, as long as the functioning of the protein is maintained. In this specification, the term “homology” can also refer to “identity”.

Homology between two amino acid sequences can be determined using well-known methods, for example, the computer program BLAST 2.0, which calculates three parameters: score, identity and similarity.

Moreover, the gene of the argT-hisJQMP cluster may be a variant which hybridizes under stringent conditions with the nucleotide sequence shown in SEQ ID NO: 1, SEQ ID NO: 3, SEQ ID NO: 5, SEQ ID NO: 7 or SEQ ID NO: 9, or a probe which can be prepared from the nucleotide sequence, under stringent conditions, provided that it encodes a functional protein. “Stringent conditions” include those under which a specific hybrid, for example, a hybrid having homology of not less than 60%, or in another example not less than 70%, or in another example not less than 80%, or in another example not less than 90%, or in another example not less than 95%, in another example not less than 98%, or in another example not less than 99%, is formed and a non-specific hybrid, for example, a hybrid having homology lower than the above, is not formed. For example, stringent conditions can be exemplified by washing one time or more, or in another example two or three times at a salt concentration of 1×SSC, 0.1% SDS, preferably 0.1×SSC, 0.1% SDS at 60° C. Duration of washing depends on the type of membrane used for blotting and, as a rule, should be what is recommended by the manufacturer. For example, the recommended duration of washing for the Hybond™ N+ nylon membrane (Amersham) under stringent conditions is 15 minutes. Washing may be performed 2 to 3 times. The length of the probe may be suitably selected depending on the hybridization conditions, and is usually 100 by to 1 kbp.

Expression of the gene of the argT-hisJQMP cluster can be attenuated by introducing mutations into the genes so that the intracellular activity of the protein encoded by the gene is decreased as compared with an unmodified strain. Such a mutation on the gene can be a replacement of one base or more to cause amino acid substitution in the protein encoded by the gene (missense mutation), introduction of a stop codon (nonsense mutation), deletion of one or two bases to cause a frame shift, insertion of a drug-resistance gene, or deletion of a part of the gene or the entire gene (Qiu, Z. and Goodman, M. F., J. Biol. Chem., 272, 8611-8617 (1997); Kwon, D. H. et al, J. Antimicrob. Chemother., 46, 793-796 (2000)). Expression of the gene of the argT-hisJQMP cluster can also be attenuated by modifying an expression regulating sequence such as the promoter, the Shine-Dalgarno (SD) sequence, etc. of each gene (WO95/34672, Carrier, T. A. and Keasling, J. D., Biotechnol Prog 15, 58-64 (1999)).

For example, the following methods may be employed to introduce a mutation by gene recombination. A mutant gene encoding a mutant protein with decreased activity can be prepared, and a bacterium to be modified can be transformed with a DNA fragment containing the mutant gene. Then, the native gene on the chromosome is replaced with the mutant gene by homologous recombination, and the resulting strain can be selected. Such gene replacement by homologous recombination can be conducted by employing a linear DNA, which is known as “Red-driven integration” (Datsenko, K. A. and Wanner, B. L., Proc. Natl. Acad. Sci. USA, 97, 12, p 6640-6645 (2000), WO2005/010175), or by methods employing a plasmid containing a temperature-sensitive replication control region (Proc. Natl. Acad. Sci., USA, 97, 12, p 6640-6645 (2000), U.S. Pat. Nos. 6,303,383 and 5,616,480). Furthermore, the introduction of a site-specific mutation by gene replacement using homologous recombination as set forth above can also be performed by using a plasmid which is unable to replicate in the host.

Expression of the target gene can also be attenuated by insertion of a transposon or an IS factor into the coding region of the gene (U.S. Pat. No. 5,175,107), or by conventional methods, such as mutagenesis treatment using UV irradiation or nitrosoguanidine (N-methyl-N′-nitro-N-nitrosoguanidine) treatment.

Inactivation of the target gene can also be performed by conventional methods, such as a mutagenesis treatment using UV irradiation or nitrosoguanidine (N-methyl-N′-nitro-N-nitrosoguanidine) treatment, site-directed mutagenesis, gene disruption using homologous recombination, or/and insertion-deletion mutagenesis (Yu, D. et al., Proc. Natl. Acad. Sci. USA, 2000, 97:12: 5978-83 and Datsenko, K. A. and Wanner, B. L., Proc. Natl. Acad. Sci. USA, 2000, 97:12: 6640-45) also called “Red-driven integration”.

Methods for preparation of plasmid DNA, digestion and ligation of DNA, transformation, selection of an oligonucleotide as a primer, and the like may be ordinary methods well-known to one skilled in the art. These methods are described, for instance, in Sambrook, J., Fritsch, E. F., and Maniatis, T., “Molecular Cloning: A Laboratory Manual, Second Edition”, Cold Spring Harbor Laboratory Press (1989).

L-Amino Acid-Producing Bacteria

The bacteria in which expression is attenuated of one or more genes encoding a lysine/arginine/ornithine transporter can be bacteria which are able to produce L-amino acids such as diaminomonocarboxylic acid, and may be used in the method in accordance with the presently disclosed subject matter.

The bacterium can be obtained by attenuating expression of the gene or genes encoding a lysine/arginine/ornithine transporter in a bacterium which inherently has the ability to produce L-amino acids such as diaminomonocarboxylic acid. Alternatively, the bacterium can be obtained by imparting the ability to produce L-amino acids such as diaminomonocarboxylic acid to a bacterium in which expression of the gene or genes encoding a lysine/arginine/ornithine transporter has already been attenuated.

L-Lysine-Producing Bacteria

Examples of L-lysine-producing bacteria or parent strains belonging to the genus Escherichia include mutants having resistance to an L-lysine analogue. The L-lysine analogue inhibits growth of bacteria belonging to the genus Escherichia, but this inhibition is fully or partially desensitized when L-lysine is present in the medium. Examples of the L-lysine analogue include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred from the W3110 strain, which was derived from Escherichia coli K-12, by replacing the wild-type lysC gene on the chromosome of the W3110 strain with a mutant lysC gene encoding a mutant aspartokinase III desensitized to feedback inhibition by L-lysine in which threonine at position 352 has been replaced with isoleucine, and conferring AEC resistance to the resulting strain (U.S. Pat. No. 5,661,012). The resulting strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains which can be used to derive bacteria which are able to produce L-lysine also include strains in which expression of one or more genes encoding an L-lysine biosynthetic enzyme are enhanced. Examples of such genes include, but are not limited to, genes encoding dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), and aspartase (aspA) (EP 1253195 A). In addition, the parent strains may have increased expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

Examples of parent strains which can be used to derive bacteria that are able to produce L-lysine also include strains having decreased or no activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase, lysine decarboxylase (U.S. Pat. No. 5,827,698), and the malic enzyme (WO2005/010175).

L-Arginine-Producing Bacteria

Examples of parent strains which can be used to derive bacteria that are able to produce L-arginine include, but are not limited to, strains belonging to the genus Escherichia, such as E. coli strain 237 (VKPM B-7925) (U.S. Patent Application 2002/058315 A1) and its derivative strains harboring mutant N-acetylglutamate synthase (Russian Patent Application No. 2001112869), E. coli strain 382 (VKPM B-7926) (EP1170358A1), an arginine-producing strain into which the argA gene encoding N-acetylglutamate synthetase is introduced therein (EP1170361A1), and the like.

Examples of parent strains which can be used to derive bacteria that can produce L-arginine also include strains in which expression of one or more genes encoding an L-arginine biosynthetic enzyme are enhanced. Examples of such genes include genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase (carAB).

L-Citrulline Producing Bacteria

Examples of L-citrulline-producing bacteria or parent strains which can be used to derive bacteria that are able to produce citrulline include, but are not limited to, strains belonging to the genus Escherichia, such as E.coli mutant N-Acetylglutamate Synthase strains 237/pMADS 11, 237/pMADS 12 and 237/pMADS 13 (RU2215783, EP1170361B1, U.S. Pat. No. 6,790,647B2).

Also, bacteria which produce citrulline can be easily obtained from any bacterium which is able to produce arginine, for example E. coli stain 382 (VKPM B-7926), by inactivation of argininosuccinate synthase encoded by the argG gene.

The phrase “inactivation of argininosuccinate synthase” can mean that the bacterium has been modified in such a way that the modified bacterium contains an inactive argininosuccinate synthase, or it can also mean that the bacterium is unable to synthesize the argininosuccinate synthase. Inactivation of argininosuccinate synthase can be performed by inactivation of the argG gene.

The phrase “inactivation of the argG gene” can mean that the modified gene encodes a completely non-functional protein. It is also possible that the modified DNA region is unable to naturally express the gene due to a deletion of a part of the gene or the whole gene, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc.

The presence or absence of the argG gene on the chromosome of a bacterium can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the argG gene can be measured by well-known methods, including SDS-PAGE followed by an immunoblotting assay (Western blotting analysis), and the like.

Expression of the argG gene can be attenuated by introducing a mutation into the gene on the chromosome so that the intracellular activity of the protein encoded by the gene is decreased as compared with an unmodified strain. Mutations which result in attenuation of expression of the gene include the replacement of one base or more to cause an amino acid substitution in the protein encoded by the gene (missense mutation), introduction of a stop codon (nonsense mutation), deletion of one or two bases to cause a frame shift, insertion of a drug-resistance gene, or deletion of a part of the gene or the entire gene (Qiu, Z. and Goodman, M. F., J. Biol. Chem., 272, 8611-8617 (1997); Kwon, D. H. et al, J. Antimicrob. Chemother., 46, 793-796 (2000)). Expression of the argG gene can also be attenuated by modifying an expression regulating sequence such as the promoter, the Shine-Dalgarno (SD) sequence, etc. (WO95/34672, Carrier, T. A. and Keasling, J. D., Biotechnol Prog 15, 58-64 (1999)).

For example, the following methods may be employed to introduce a mutation by gene recombination. A mutant gene encoding a mutant protein with decreased activity is prepared, and the bacterium is transformed with a DNA fragment containing the mutant gene. Then, the native gene on the chromosome is replaced with the mutant gene by homologous recombination, and the resulting strain is selected. Gene replacement or disruption using homologous recombination can be conducted by employing a linear DNA, which is known as “Red-driven integration” (Datsenko, K. A. and Wanner, B. L., Proc. Natl. Acad. Sci. USA, 97, 12, p 6640-6645 (2000)), or by employing a plasmid containing a temperature-sensitive replication origin (U.S. Pat. No. 6,303,383 or JP 05-007491A). Furthermore, site-specific mutation by gene substitution can also be incorporated using homologous recombination such as set forth above using a plasmid that is unable to replicate in the host.

Expression of the gene can also be attenuated by inserting a transposon or an IS factor into the coding region of the gene (U.S. Pat. No. 5,175,107), or by conventional methods, such as by mutagenesis with UV irradiation or nitrosoguanidine (N-methyl-N′-nitro-N-nitrosoguanidine).

L-Ornithine Producing Bacteria

Bacteria which are able to produce ornithine can be easily obtained from any arginine-producing bacteria, for example E. coli strain 382 (VKPM B-7926), by inactivation of ornithine carbamoyltransferase encoded by both the argF and argI genes. Inactivation of ornithine carbamoyltransferase can be performed in the same way as described above.

2. Method of the Present Invention

Exemplary methods in accordance with the presently disclosed subject matter can include production of an L-amino acid by cultivating a bacterium in a culture medium to produce and excrete the L-amino acid into the medium, and collecting the L-amino acid from the medium.

The cultivation, collection, and purification of an L-amino acid from the medium and the like may be performed in a manner similar to conventional fermentation methods wherein an amino acid is produced using a bacterium.

The medium used for culture may be either a synthetic or natural medium, so long as the medium includes a carbon source and a nitrogen source and minerals and, if necessary, appropriate amounts of nutrients which the chosen bacterium requires for growth. The carbon source may include various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation can be performed under aerobic conditions, such as by shaking, and/or by stirring with aeration, at a temperature of 20 to 40° C., or in another example 30 to 38° C. The pH of the culture is usually between 5 and 9, or in another example between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids such as cells can be removed from the liquid medium by centrifugation or membrane filtration, and then the L-amino acid can be collected and purified by ion-exchange, concentration, and/or crystallization methods.

### EXAMPLES

The present invention will be more concretely explained below with reference to the following non-limiting Examples.

### Example 1

### Construction of a Strain with an Inactivated argT-hisJQMP Cluster

### 1. Deletion of the argT-hisJQMP Cluster.

A strain in which the argT-hisJQMP cluster is deleted was constructed by the method initially developed by Datsenko, K. A. and Wanner, B. L. (Proc. Natl. Acad. Sci. USA, 2000, 97(12), 6640-6645) called “Red-driven integration”. The DNA fragment containing the Cm^{R} marker encoded by the cat gene was obtained by PCR, using primers P1 (SEQ ID NO: 11) and P2 (SEQ ID NO: 12), and plasmid pMW118-attL-Cm-attR as the template (WO 05/010175). Primer P1 contains both a region complementary to the region located at the 5′ end of the argT gene and a region complementary to the attR region. Primer P2 contains both a region complementary to the region located at the 3′ end of the hisP gene and a region complementary to the attL region. The conditions for PCR were as follows: denaturation step for 3 min at 95° C.; profile for two first cycles: 1 min at 95° C., 30 sec at 50° C., 40 sec at 72° C.; profile for the last 25 cycles: 30 sec at 95° C., 30 sec at 54° C., 40 sec at 72° C.; final step: 5 min at 72° C.

A 1.6 kbp PCR product was obtained and purified in agarose gel and was used for electroporation of the E. coli strain MG1655 (ATCC 700926), which contains the plasmid pKD46. The plasmid pKD46 (Datsenko, K. A. and Wanner, B. L., Proc. Natl. Acad. Sci. USA, 2000, 97:12:6640-45) contains a temperature-sensitive replication origin, and includes a 2,154 nucleotide DNA fragment of phage λ (nucleotide positions 31088 to 33241, GenBank accession no. J02459), as well as genes of the λ Red homologous recombination system (γ, β, exo genes) under the control of the arabinose-inducible P_{araB} promoter. The plasmid pKD46 is necessary for integration of the PCR product into the chromosome of strain MG1655. The strain MG1655 can be obtained from the American Type Culture Collection. (P.O. Box 1549 Manassas, Va. 20108, U.S.A.).

Electrocompetent cells were prepared as follows: E. coli MG1655/pKD46 was grown overnight at 30° C. in LB medium containing ampicillin (100 mg/l), and the culture was diluted 100 times with 5 ml of SOB medium (Sambrook et al, “Molecular Cloning: A Laboratory Manual, Second Edition”, Cold Spring Harbor Laboratory Press, 1989) containing ampicillin and L-arabinose (1 mM). The cells were grown with aeration at 30° C. to an OD₆₀₀ of ≈0.6 and then were made electrocompetent by concentrating 100-fold and washing three times with ice-cold deionized H₂O. Electroporation was performed using 70 μl of cells and ≈100 ng of the PCR product. Cells after electroporation were incubated with 1 ml of SOC medium (Sambrook et al, “Molecular Cloning: A Laboratory Manual, Second Edition”, Cold Spring Harbor Laboratory Press, 1989) at 37° C. for 2.5 hours and then were plated onto L-agar containing chloramphenicol (30 μg/ml) and grown at 37° C. to select Cm^{R} recombinants. Then, to eliminate the pKD46 plasmid, two passages on L-agar with Cm at 42° C. were performed and the obtained colonies were tested for sensitivity to ampicillin.

Verification of Deletion of the argT-hisJQMP Cluster by PCR

The recombinants in which the argT-hisJQMP cluster had been deleted and marked with the Cm resistance gene were verified by PCR. Locus-specific primers P3 (SEQ ID NO: 13) and P4 (SEQ ID NO: 14) were used in PCR for the verification. Conditions for PCR verification were as follows: denaturation step for 3 min at 94° C.; profile for 30 cycles: 30 sec at 94° C., 30 sec at 54° C., 1 min at 72° C.; final step: 7 min at 72° C. The PCR product obtained in the reaction with the cells of the recombinant strain as the template was 1650 bp in length. The recombinant strain was named MG1655 Δ argT-hisJQMP::cat.

### Example 2

### Production of L-arginine by E. coli 382ΔargT-hisP

To test the effect of inactivation of the argT-hisJQMP cluster on L-arginine production, DNA fragments from the chromosome of the above-described E. coli MG1655 ΔargT-hisP::cat were transferred to the arginine-producing E. coli strain 382 by P1 transduction (Miller, J. H. Experiments in Molecular Genetics, Cold Spring Harbor Lab. Press, 1972, Plainview, N.Y.) to obtain the strain 382ΔargT-hisP. The strain 382 was deposited in the Russian National Collection of Industrial Microorganisms (VKPM) (Russia, 117545 Moscow, 1 Dorozhny proezd, 1) on Apr. 10, 2000 under accession number VKPM B-7926 and then converted to a deposit under the Budapest Treaty on May 18, 2001.

Both E. coli strains, 382 and 382ΔargT-hisP, were separately cultivated with shaking at 37° C. for 18 hours in 3 ml of nutrient broth, and 0.3 ml of the obtained cultures were inoculated into 2 ml of a fermentation medium in 20×200-mm test tubes and cultivated at 32° C. for 48 hours on a rotary shaker.

After the cultivation, the amount of L-arginine which accumulates in the medium was determined by paper chromatography using the following mobile phase:butanol:acetic acid:water=4:1:1 (v/v). A solution of ninhydrin (2%) in acetone was used as a visualizing reagent. A spot containing L-arginine was cut out, L-arginine was eluted with a 0.5% water solution of CdCl₂, and the amount of L-arginine was estimated spectrophotometrically at 540 nm. The results of eight independent test tube fermentations are shown in Table 1. As follows from Table 1, the strain 382ΔargT-hisP produced a larger amount of L-arginine, as compared with 382.

The composition of the fermentation medium (g/l) was as follows:

| | |
|---|---|
| Glucose | 48.0 |
| (NH₄)₂SO₄ | 35.0 |
| KH₂PO₄ | 2.0 |
| MgSO₄ · 7H₂O | 1.0 |
| Thiamine HCl | 0.0002 |
| Yeast extract | 1.0 |
| L-isoleucine | 0.1 |
| CaCO₃ | 5.0 |

Glucose and magnesium sulfate were sterilized separately. CaCO₃ was dry-heat sterilized at 180° C. for 2 hours. The pH was adjusted to 7.0.

TABLE 1

| | | Amount of |
|---|---|---|
| Strain | OD₅₄₀ | L-arginine, g/l |
| 382 | 11.3 ± 1.9 | 10.7 ± 1.0 |
| 382ΔargT-hisP | 13.2 ± 1.0 | 11.6 ± 0.7 |

### Example 3

### Production of L-Lysine by E. coli AJ11442-ΔargT-hisP

To test the effect of inactivation of the argT-hisJQMP cluster on lysine production, DNA fragments from the chromosome of the above-described E. coli strain MG1655 ΔargT-hisP::cat can be transferred to the lysine-producing E. coli strain AJ11442 by P1 transduction (Miller, J. H. Experiments in Molecular Genetics, Cold Spring Harbor Lab. Press, 1972, Plainview, N.Y.) to obtain the AJ11442ΔargT-hisP strain. The strain AJ11442 was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on May 1, 1981 and received an accession number of FERM P-5084. Then, it was converted to an international deposit under the provisions of the Budapest Treaty on Oct. 29, 1987, and received an accession number of FERM BP-1543.

Both E. coli strains, AJ11442 and AJ11442ΔargT-hisP, can be cultured in L-medium containing streptomycin (20 mg/l) at 37° C., and 0.3 ml of the obtained culture can be inoculated into 20 ml of the fermentation medium containing the required drugs in a 500-ml flask. The cultivation can be carried out at 37° C. for 16 h by using a reciprocal shaker at the agitation speed of 115 rpm. After the cultivation, the amounts of L-lysine and residual glucose in the medium can be measured by a known method (Biotech-analyzer AS210 manufactured by Sakura Seiki Co.). Then, the yield of L-lysine can be calculated relative to consumed glucose for each of the strains.

The composition of the fermentation medium (g/l) is as follows:

| | |
|---|---|
| Glucose | 40 |
| (NH₄)₂SO₄ | 24 |
| K₂HPO₄ | 1.0 |
| MgSO₄ 7H₂O | 1.0 |
| FeSO₄ 7H₂O | 0.01 |
| MnSO₄ 5H₂O | 0.01 |
| Yeast extract | 2.0 |

The pH is adjusted to 7.0 by KOH and the medium is autoclaved at 115° C. for 10 min. Glucose and MgSO₄ 7H₂O are sterilized separately. CaCO₃ is dry-heat sterilized at 180° C. for 2 hours and added to the medium for a final concentration of 30 g/l.

### Example 4

### Production of Citrulline by E. coli Strain 382 ilvA⁺ ΔargG ΔargT-hisP

To test the effect of inactivation of the argT-hisJQMP cluster on citrulline production, the citrulline-producing strain 382 ilvA⁺ ΔargG was constructed in the following way.

The strain 382 ilvA⁺ was obtained from the arginine-producing strain 382 (VKPM B-7926) by introducing a wild type ilvA gene from E.coli K12 strain by P1 transduction. Clones 382 ilvA⁺ were selected as good growing colonies on minimal agar plates. After that, the citrulline-producing strain 382 ilvA⁺ ΔargG was obtained by deleting the argG gene on the chromosome of the 382 ilvA⁺ strain by the method initially developed by Datsenko, K. A. and Wanner, B. L. (Proc. Natl. Acad. Sci. USA, 2000, 97(12), 6640-6645) called “Red-driven integration”.

The DNA fragment containing the Cm^{R} marker encoded by the cat gene was obtained by PCR, using primers P5 (SEQ ID NO: 15) and P6 (SEQ ID NO: 16), and plasmid pMW118-attL-Cm-attR as the template (WO 05/010175). Primer P5 contains both a region complementary to the region located at the 5′ end of the argG gene and a region complementary to the attR region. Primer P6 contains both a region complementary to the region located at the 3′ end of the argG gene and a region complementary to the attL region. The conditions for PCR were as follows: denaturation step for 3 min at 95° C.; profile for two first cycles: 1 min at 95° C., 30 sec at 50° C., 40 sec at 72° C.; profile for the last 25 cycles: 30 sec at 95° C., 30 sec at 54° C., 40 sec at 72° C.; final step: 5 min at 72° C.

A 1.85 kbp PCR product was obtained and purified in agarose gel and was used for electroporation of the E. coli strain 382 ilvA⁺, which was previously transformed with the plasmid pKD46 which is necessary for integration of the PCR product into the bacterial chromosome. Electrocompetent cells were prepared as described in Example 1. Electroporation was performed using 70 μl of cells and ≈100 ng of the PCR product. Cells after electroporation were incubated and cured from the pKD46 plasmid as described in Example 1.

The recombinants in which the argG gene was deleted and marked with the Cm resistance gene were verified by PCR. Locus-specific primers P7 (SEQ ID NO: 17) and P8 (SEQ ID NO: 18) were used in PCR for the verification. Conditions for PCR verification were as follows: denaturation step for 3 min at 94° C.; profile for 30 cycles: 30 sec at 94° C., 30 sec at 54° C., 1 min at 72° C.; final step: 7 min at 72° C. The PCR product obtained in the reaction with the cells of the recombinant strain as the template was 1350 bp in length. The recombinant strain was named 382 ilvA⁺ ΔargG::cat.

Then, the Cm resistance gene (cat gene) was eliminated from the chromosome of the E. coli 382 ilvA⁺ ΔargG::cat strain using the int-xis system. For that purpose E. coli strain 382 ilvA⁺ ΔargG::cat was transformed with pMW-intxis-ts plasmid (WO 05/010175). The plasmid pMW-intxis-ts carries the gene encoding integrase (Int) of λ phage, and the gene encoding excisionase (Xis), and has temperature-sensitive replicability. Transformant clones were selected on the LB-medium containing 100 μg/ml of ampicillin. Plates were incubated overnight at 30° C. Transformant clones were cured from the cat gene and pMW-intxis-ts plasmid by spreading the separate colonies at 37° C. (at this temperature repressor CIts is partially inactivated and transcription of the int/xis genes is derepressed) followed by selection of Cm^{S}Ap^{R} variants. Elimination of the cat gene from the chromosome of the strain was verified by PCR. Locus-specific primers P7 (SEQ ID NO: 17) and P8 (SEQ ID NO: 18) were used in PCR for the verification. Conditions for PCR verification were as described above. The PCR product obtained in reaction with cells having the eliminated cat gene as a template was ˜0.44 kbp in length. Thus, the citrulline-producing strain 382 ilvA⁺ ΔargG was obtained.

To test the effect of inactivation of the argT-hisJQMP cluster on citrulline production, DNA fragments from the chromosome of the above-described E. coli strain MG1655 ΔargT-hisP::cat was transferred to the E. coli citrulline-producing strain 382 ilvA⁺ ΔargG by P1 transduction (Miller, J. H. Experiments in Molecular Genetics, Cold Spring Harbor Lab. Press, 1972, Plainview, N.Y.) to obtain the 382 ilvA⁺ ΔargGΔargT-hisP strain.

Both E. coli strains, 382 ilvA⁺ ΔargG and 382 ilvA⁺ ΔargG ΔargT-hisP, were separately cultivated with shaking at 37° C. for 18 hours in 3 ml of nutrient broth, and 0.3 ml of the obtained cultures were inoculated into 2 ml of a fermentation medium in 20×200-mm test tubes and cultivated at 32° C. for 48 hours on a rotary shaker.

After the cultivation, the amount of citrulline which accumulates in the medium was determined by paper chromatography using the following mobile phase:butanol:acetic acid:water=4:1:1 (v/v). A solution of ninhydrin (2%) in acetone can be used as a visualizing reagent. A spot containing citrulline was cut out, citrulline was eluted with 0.5% water solution of CdCl₂, and the amount of citrulline was estimated spectrophotometrically at 540 nm. The results of eight independent test tube fermentations are shown in Table 2. As follows from Table 2, the strain 382 ilvA⁺ ΔargGΔargT-hisP produced a larger amount of L-citrulline, as compared with 382 ilvA⁺ ΔargG.

The composition of the fermentation medium (g/l) can be as follows:

| | |
|---|---|
| Glucose | 48.0 |
| (NH₄)₂SO₄ | 35.0 |
| KH₂PO₄ | 2.0 |
| MgSO₄ · 7H₂O | 1.0 |
| Thiamine HCl | 0.0002 |
| Yeast extract | 1.0 |
| L-arginine | 0.1 |
| CaCO₃ | 5.0 |

Glucose and magnesium sulfate are sterilized separately. CaCO₃ is dry-heat sterilized at 180° C. for 2 hours. The pH is adjusted to 7.0.

TABLE 2

| | | Amount of |
|---|---|---|
| Strain | OD₅₄₀ | L-citrulline, g/l |
| 382 ilvA⁺ ΔargG | 12.1 ± 0.3 | 4 ± 0.3 |
| 382 ilvA⁺ ΔargG | 10.4 ± 0.7 | 4.3 ± 0.2 |
| ΔargT-hisP | | |

### Example 5

### Production of Ornithine by E. coli Strain 382 ΔargFΔargI ΔargT-hisP

To test the effect of inactivation of the argT-hisJQMP cluster on ornithine production, DNA fragments from the chromosome of the above-described E. coli strain MG1655 ΔargT-hisP::cat can be transferred to the E. coli ornithine producing strain 382ΔargFΔargI by P1 transduction (Miller, J. H. Experiments in Molecular Genetics, Cold Spring Harbor Lab. Press, 1972, Plainview, N.Y.) to obtain 382 ΔargFΔargIΔargT-hisP strain. The strain 382ΔargFΔargI can be obtained by consecutive deletion of the argF and argI genes on the chromosome of the 382 strain (VKPM B-7926) by the method initially developed by Datsenko, K. A. and Wanner, B. L. (Proc. Natl. Acad. Sci. USA, 2000, 97(12), 6640-6645) called “Red-driven integration”. According to this procedure, two pairs of PCR primers homologous to both the region adjacent to the argF or argI gene and the gene which confers antibiotic resistance in the template plasmid can be constructed. The plasmid pMW118-attL-Cm-attR (WO 05/010175) can be used as the template in the PCR reaction.

Both E. coli strains, 382ΔargFΔargI and 382ΔargFΔargIΔargT-hisP, can be separately cultivated with shaking at 37° C. for 18 hours in 3 ml of nutrient broth, and 0.3 ml of the obtained cultures were inoculated into 2 ml of a fermentation medium in 20×200-mm test tubes and cultivated at 32° C. for 48 hours on a rotary shaker.

After the cultivation, the amount of ornithine which accumulates in the medium can be determined by paper chromatography using the following mobile phase:butanol:acetic acid:water=4:1:1 (v/v). A solution of ninhydrin (2%) in acetone can be used as a visualizing reagent. A spot containing ornithine can be cut out, ornithine can be eluted with 0.5% water solution of CdCl₂, and the amount of ornithine can be estimated spectrophotometrically at 540 nm.

The composition of the fermentation medium (g/l) can be as follows:

| | |
|---|---|
| Glucose | 48.0 |
| (NH₄)₂SO₄ | 35.0 |
| KH₂PO₄ | 2.0 |
| MgSO₄ · 7H₂O | 1.0 |
| Thiamine HCl | 0.0002 |
| Yeast extract | 1.0 |
| L-isoleucine | 0.1 |
| L-arginine | 0.1 |
| CaCO₃ | 5.0 |

Glucose and magnesium sulfate are sterilized separately. CaCO₃ is dry-heat sterilized at 180° C. for 2 hours. The pH is adjusted to 7.0.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL APPLICABILITY

According to the present invention, production of L-amino acid such as L-lysine, L-arginine, ornithine, and citrulline by a bacterium of Enterobacteriaceae family can be improved.

1. An L-amino acid-producing bacterium of the Enterobacteriaceae family, wherein said bacterium has been modified to attenuate expression of one or more genes encoding a lysine/arginine/ornithine transporter.

2. The bacterium according to claim 1, wherein said one or more genes encoding a lysine/arginine/ornithine transporter is/are one or more genes of argT-hisJQMP cluster.

3. The bacterium according to claim 1, wherein expression of said one or more genes encoding a lysine/arginine/ornithine transporter is/are attenuated by inactivation of said one or more genes.

4. The bacterium according to claim 2, wherein said one or more genes of the argT-hisJQMP cluster is/are inactivated.

5. The bacterium according to claim 1, wherein said bacterium belongs to genus Escherichia.

6. The bacterium according to claim 5, wherein said bacterium is Escherichia coli.

7. The bacterium according to claim 1, wherein said bacterium belongs to genus Pantoea.

8. The L-amino acid-producing bacterium according to claim 1, wherein said L-amino acid is a diaminomonocarboxylic acid.

9. The L-amino acid-producing bacterium according to claim 8, wherein said diaminomonocarboxylic acid is selected from the group consisting of L-lysine, L-arginine, L-ornithine, and L-citrulline.

10. A method for producing an L-amino acid comprising:
cultivating the bacterium according to claim 1 in a medium, and
collecting said L-amino acid from the medium.

11. The method according to claim 10, wherein said L-amino acid is a diaminomonocarboxylic acid.

12. The method according to claim 11, wherein said diaminomonocarboxylic acid is selected from the group consisting of L-lysine, L-arginine, ornithine, and citrulline.


SEQUENCE LISTING (18 sequences)

SEQ ID NO: 1 - DNA, 783 bp, Escherichia coli
  CDS (1)..(783)
atgaagaagtcgattctcgctctgtctttgttagtcggtctctccacagcggcttccagctatgcggcgctaccggagacggtacgtatcggaaccgataccacctacgcaccgttctcatcgaaagatgctaaaggtgattttgttggctttgatatcgatctcggtaacgagatgtgcaaacggatgcaggtgaaatgtacctgggttgccagtgactttgacgcgctgatcccctcactgaaagcgaaaaaaatcgacgctattatttcgtcgctttccattaccgataaacgtcagcaggagattgccttctccgacaagctgtacgccgcagattctcgtttgattgcggccaaaggttcaccgattcagccaacgctggattcactgaaaggtaaacatgttggtgtgctgcagggatcaacccaggaagcttacgctaacgagacctggcgtagtaaaggcgtggatgtggtggcctatgccaaccaggatttggtctattccgatctggctgcaggacgtctggatgctgcgttacaagatgaagttgctgccagcgaaggattcctcaagcaacctgctggtaaagatttcgcctttgctggctcatcagtaaaagacaaaaaatacttcggtgatggcaccggtgtagggctacgtaaagatgatgctgaactgacggctgccttcaataaggcgcttggcgagctgcgtcaggacggcacctacgacaagatggcgaaaaagtatttcgactttaatgtctacggtgactga

SEQ ID NO: 2 - protein, 260 aa, Escherichia coli
MKKSILALSLLVGLSTAASSYAALPETVRIGTDTTYAPFSSKDAKGDFVGFDIDLGNEMCKRMQVKCTWVASDFDALIPSLKAKKIDAIISSLSITDKRQQEIAFSDKLYAADSRLIAAKGSPIQPTLDSLKGKHVGVLQGSTQEAYANETWRSKGVDVVAYANQDLVYSDLAAGRLDAALQDEVAASEGFLKQPAGKDFAFAGSSVKDKKYFGDGTGVGLRKDDAELTAAFNKALGELRQDGTYDKMAKKYFDFNVYGD

SEQ ID NO: 3 - DNA, 783 bp, Escherichia coli
  CDS (1)..(783)
atgaaaaaactggtgctatcgctctctctggttctggccttctccagcgcaactgcggcgtttgctgcgattccgcaaaacatccgcatcggtaccgacccgacctatgcgccatttgaatcaaaaaattcacaaggcgaactggttggcttcgatatcgatctggcaaaggaattatgcaaacgcatcaatacgcaatgtacgtttgtcgaaaatccgctggatgcgttaatcccgtccttaaaagcgaagaagattgacgccatcatgtcatcgctttccattacggaaaaacgtcagcaagaaatagccttcaccgacaaactgtacgctgccgattctcgtttggtggtggcgaaaaattctgacattcagccgacagtcgagtcgctgaaaggcaaacgggtaggcgtattgcagggcaccacccaggagacgttcggtaatgaacattgggcaccaaaaggcattgaaatcgtctcgtatcaggggcaggacaacatttattctgacctgactgccggacgtattgatgccgcgttccaggatgaggtcgctgccagcgaaggtttcctcaaacaacctgtcggtaaagattacaaattcggtggcccgtctgttaaagatgaaaaactgtttggcgtagggaccggcatgggcctgcgtaaagaagataacgaactgcgcgaagcactgaacaaagcctttgccgaaatgcgcgctgacggtacttacgagaaattagcgaaaaagtacttcgattttgatgtttatggtggctaa

SEQ ID NO: 4 - protein, 260 aa, Escherichia coli
MKKLVLSLSLVLAFSSATAAFAAIPQNIRIGTDPTYAPFESKNSQGELVGFDIDLAKELCKRINTQCTFVENPLDALIPSLKAKKIDAIMSSLSITEKRQQEIAFTDKLYAADSRLVVAKNSDIQPTVESLKGKRVGVLQGTTQETFGNEHWAPKGIEIVSYQGQDNIYSDLTAGRIDAAFQDEVAASEGFLKQPVGKDYKFGGPSVKDEKLFGVGTGMGLRKEDNELREALNKAFAEMRADGTYEKLAKKYFDFDVYGG

SEQ ID NO: 5 - DNA, 687 bp, Escherichia coli
  CDS (1)..(687)
atgttgtatgggttttcaggtgttattttacagggtgcgctcgtcacgctggagctggctatcagctctgtagtgctcgctgtaatcatcggtttaattggcgctggcggtaagctctcgcaaaatcggctttcggggctgattttcgaagggtacaccacgctgattcgtggcgtgccggacttagtgttgatgctgctgattttctacggtttgcagattgcgctaaacacggtgacggaggcgatgggcgtcgggcagattgatatcgatccgatggtcgctggtattatcactctcggttttatctacggtgcttattttaccgaaacgttccgtggtgcttttatggcagtgccgaaaggacatatagaggcggcgacggcgttcggttttactcgtgggcaagtgtttcggcggatcatgtttccgtcgatgatgcgttacgccctgccaggcattggcaacaactggcaggtgatcctcaaatctaccgcactggtttcgttactcggcctggaagatgtggtcaaagccacccaactggcaggcaaaagtacctgggaaccgttctatttcgccatcgtctgtggcgtgatttacctggttttcaccactgtttccaatggtgtgctgctgttccttgagcgccgctactccgtgggtgtgaagagggctgacctgtga

SEQ ID NO: 6 - protein, 228 aa, Escherichia coli
MLYGFSGVILQGALVTLELAISSVVLAVIIGLIGAGGKLSQNRLSGLIFEGYTTLIRGVPDLVLMLLIFYGLQIALNTVTEAMGVGQIDIDPMVAGIITLGFIYGAYFTETFRGAFMAVPKGHIEAATAFGFTRGQVFRRIMFPSMMRYALPGIGNNWQVILKSTALVSLLGLEDVVKATQLAGKSTWEPFYFAIVCGVIYLVFTTVSNGVLLFLERRYSVGVKRADL

SEQ ID NO: 7 - DNA, 717 bp, Escherichia coli
  CDS (1)..(717)
gtgatcgaaatcttacatgaatactggaaaccgctgctgtggaccgacggttatcgctttactggtgtggcgatcactctgtggctgcttattttgtcggtagtgataggcggagtcctggcgctgtttctggcgattggtcgtgtctccagtaataaatacatccagtttccaatctggttatttacctatatttttcgcggtacgccgctgtatgttcagttgctggtgttctattccggcatgtacacgcttgagattgttaagggaaccgaattccttaacgctttcttccgcagtggcctgaactgtaccgtgctggcgctgacgcttaacacctgcgcttacactaccgagatttttgctggggcaatccgttcggttccgcatggggaaattgaagccgccagagcctatggcttctcgacttttaaaatgtatcgctgcattattttgccttctgcgctgcgtattgcgttaccggcatacagcaacgaagtgatcctgatgctgcactctactgcgttggcatttactgccacggtgccggatctgctgaaaatagcccgcgatattaacgccgccacgtatcaaccttttaccgccttcggcattgccgcggtgctctatttaatcatctcttatgtcctgatcagcctctttcgcagagcggaaaaacgctggttgcagcatgtgaaaccttcttcaacgcactga

SEQ ID NO: 8 - protein, 238 aa, Escherichia coli
VIEILHEYWKPLLWTDGYRFTGVAITLWLLILSVVIGGVLALFLAIGRVSSNKYIQFPIWLFTYIFRGTPLYVQLLVFYSGMYTLEIVKGTEFLNAFFRSGLNCTVLALTLNTCAYTTEIFAGAIRSVPHGEIEAARAYGFSTFKMYRCIILPSALRIALPAYSNEVILMLHSTALAFTATVPDLLKIARDINAATYQPFTAFGIAAVLYLIISYVLISLFRRAEKRWLQHVKPSSTH

SEQ ID NO: 9 - DNA, 774 bp, Escherichia coli
  CDS (1)..(774)
atgtccgagaataaattaaacgttatcgatttgcacaaacgctacggcgaacatgaagtgctgaaaggggtatcactgcaagcgaatgccggagatgtaataagcatcatcggatcgtcgggatcggggaaaagtacctttctgcgctgcattaacttcctcgaaaaaccgagtgaagggtcgatcgtggtcaatggccagacgatcaatctggtgcgcgacaaagacggtcaactcaaagtcgccgataaaaatcaactgcgcttactgcgcacacgcctgacgatggtattccagcacttcaatctctggagccatatgacggtgctggaaaacgtcatggaagcgccgattcaggtgttgggcctgagcaagcaggaagcgcgcgagcgggcggtgaagtatctggcaaaagtcgggatagacgaacgtgcgcaggggaaatatccggtgcatctttccggcggtcagcaacagcgtgtttctatcgcgcgggcgctggcgatggaaccggaagttttactgtttgatgaacctacctcggcgctcgatcctgaactggtaggcgaagtgttgcgtattatgcagcaactggcagaagaggggaaaaccatggtggtagtgactcacgaaatgggctttgctcgccatgtttctactcatgtcattttcctccatcaggggaaaatagaagaagagggcgcgccggagcagttatttggcaacccgcaaagccctcgtctgcaacggttccttaagggatcgctgaaataa

SEQ ID NO: 10 - protein, 257 aa, Escherichia coli
MSENKLNVIDLHKRYGEHEVLKGVSLQANAGDVISIIGSSGSGKSTFLRCINFLEKPSEGSIVVNGQTINLVRDKDGQLKVADKNQLRLLRTRLTMVFQHFNLWSHMTVLENVMEAPIQVLGLSKQEARERAVKYLAKVGIDERAQGKYPVHLSGGQQQRVSIARALAMEPEVLLFDEPTSALDPELVGEVLRIMQQLAEEGKTMVVVTHEMGFARHVSTHVIFLHQGKIEEEGAPEQLFGNPQSPRLQRFLKGSLK

SEQ ID NO: 11 - DNA, 64 bp, Artificial Sequence
  Primer P1
gttatgtatgaagaagtcgattctcgctctgtctttcgctcaagttagtataaaaaagctgaac

SEQ ID NO: 12 - DNA, 64 bp, Artificial Sequence
  Primer P2
tactccggcagcgttagccaccggagaggaaattattgaagcctgcctttttatactaagttgg

SEQ ID NO: 13 - DNA, 20 bp, Artificial Sequence
  Primer P3
tgtcgtcaagatggcataag

SEQ ID NO: 14 - DNA, 21 bp, Artificial Sequence
  Primer P4
aacacccaatgcagcaggcag

SEQ ID NO: 15 - DNA, 64 bp, Artificial Sequence
  Primer P5
tcagttaattaagcagggtgttattttatgacgacgcgctcaagttagtataaaaaagctgaac

SEQ ID NO: 16 - DNA, 64 bp, Artificial Sequence
  Primer P6
gttgatgtcgaattactggcctttgttttccagatttgaagcctgcctttttatactaagttgg

SEQ ID NO: 17 - DNA, 27 bp, Artificial Sequence
  Primer P7
tagtcgactgcaggcattatagtgatc

SEQ ID NO: 18 - DNA, 30 bp, Artificial Sequence
  Primer P8
attctagaagcttggatgcaaaccatgatg